(19) United States
(12) Patent Application Publication
(10) Pub. No.: US 2026/0219177 A1
(43) Pub. Date: Jul. 30, 2026

(54) APPARATUS AND SYSTEM FOR MEASURING SOIL RESPIRATION

(71) Applicant: STELLENBOSCH UNIVERSITY, Stellenbosch (ZA)

(72) Inventors: Gideon WOLFAARDT, Stellenbosch (ZA); Kyle Brent KLOPPER, Stellenbosch (ZA)

(21) Appl. No.: 19/141,735

(22) PCT Filed: Dec. 20, 2023
(86) PCT No.: PCT/ZA2023/050075

§ 371 Date: Jun. 20, 2025

(30) Foreign Application Priority Data
Dec. 23, 2022 (GB) 2219626.5

(51) Int. Cl. G01N 21/3504; G01N 33/24

(52) U.S. Cl. CPC G01N 21/3504; G01N 33/24

(57) ABSTRACT

An apparatus and system for measuring a concentration of a gas within soil are provided. The apparatus includes a tubular housing configured to extend beneath the soil and a gas sensor. The tubular housing includes an outer protective sleeve with perforations therein, a support structure within the outer protective sleeve defining an inner chamber and a gas permeable membrane carried by the support structure and configured to prevent ingress of soil or liquid into the inner chamber. The gas sensor is in communication with the inner chamber to measure a concentration of the gas in the inner chamber.

### CROSS-REFERENCE TO RELATED APPLICATIONS

This application claims priority from United Kingdom patent application number 2219626.5 filed on 23 Dec. 2022, which is incorporated by reference herein.

### FIELD OF THE INVENTION

This invention relates to an apparatus and system for measuring concentration of a gas, such as carbon dioxide, within soil. In particular, it relates to an apparatus and system for continuously monitoring soil respiration due to microbial, microfauna and plant metabolic activity in soil to allow for precision agriculture.

### BACKGROUND TO THE INVENTION

Soil respiration is an important indicator of soil health, reflecting the chemical and physical conditions within the soil environment. Contemporary agriculture increasingly focuses on soil health through Precision Agriculture (PA) which is farm management involving information technology (IT) to ensure that crops and soil receive exactly what they need for optimum health and productivity. Soil aeration and moisture control are imperative for soil health and require both mechanical interventions such as cultivation and chemical interventions such as the application of soil conditioners.

Diffuse pollution from agriculture is the result of excessive application of both fertilizer and irrigation. It is estimated that in worse case scenarios, as much of 50% of the fertilizers applied leaches past the root zone and a large portion may end up in the groundwater, polluting aquifers. Given the increasing cost of fertilizers, there is also a significant financial reason to apply precision agriculture.

Accordingly, there is a need for inexpensive and low maintenance apparatuses and systems that monitor the effects and effectiveness of agricultural activities, including irrigation and fertilizer application, to allow for precision agriculture and avoid contamination of water sources by diffuse pollution from agriculture.

The preceding discussion of the background to the invention is intended only to facilitate an understanding of the present invention. It should be appreciated that the discussion is not an acknowledgment or admission that any of the material referred to was part of the common general knowledge in the art as at the priority date of the application.

### SUMMARY OF THE INVENTION

In accordance with an aspect of the invention there is provided an apparatus for measuring concentration of a gas within soil comprising:
a tubular housing configured to extend beneath the soil surface, the tubular housing including:
   an outer protective sleeve with perforations therein;
   a support structure within the outer protective sleeve and defining an inner chamber;
   a gas permeable membrane carried by the support structure and configured to prevent ingress of soil or liquid into the inner chamber; and
a gas sensor in communication with the inner chamber to measure a concentration of the gas in the inner chamber.

The tubular housing may include an extension with a predetermined length, wherein the predetermined length is selected for monitoring gasses in a soil zone of interest.

The apparatus may include a compartment to contain the sensor.

The extension may space the compartment and the inner chamber apart by a predetermined distance. The predetermined distance may be selected for monitoring gasses in a soil zone of interest. The extension may have an outer wall that is not gas permeable. The extension may be in fluid communication with the inner chamber and configured to allow gas diffusion from the inner chamber to the compartment.

The apparatus may include a pump configured to move a carrier gas through the inner chamber to transport gas in the inner chamber to the gas sensor. The support structure may include a conduit connected to the pump and wherein the conduit is configured to allow the flow of the carrier gas into the inner chamber.

The gas permeable membrane may be a carbon dioxide permeable silicone membrane.

The gas sensor may be configured to measure carbon dioxide (CO₂) levels. The apparatus may include a processing module configured to receive a signal from the sensor and process the signal to monitor the levels of carbon dioxide in the soil. The gas sensor may be a nondispersive infrared (NDIR) CO₂ sensor. The gas sensor may be an IoT sensor in wireless data communication with a network.

The apparatus may include a spiked end for insertion into soil.

In accordance with another aspect of the invention there is provided a system for monitoring soil respiration within soil comprising:
the apparatus as defined above; and
a controller configured to be in data communication with the gas sensor of the apparatus to continuously monitor the soil respiration measured.

Embodiments of the invention will now be described, by way of example only, with reference to the accompanying drawings.

### BRIEF DESCRIPTION OF THE DRAWINGS

In the Drawings:

FIG. 1 is an exploded three-dimensional view of a first embodiment of an apparatus for measuring soil respiration in soil;

FIG. 2 is an exploded front perspective view of the apparatus of FIG. 1;

FIG. 3 is an exploded side perspective view of the apparatus of FIG. 1;

FIG. 4 is a schematic diagram which illustrates a second embodiment of an apparatus for measuring soil respiration in soil;

FIG. 5 is an exploded three-dimensional view of the apparatus of FIG. 4;

FIG. 6 is an exploded side perspective view of the apparatus of FIG. 4;

FIG. 7 is a schematic diagram which illustrates a third embodiment of an apparatus for measuring soil respiration in soil;

FIG. 8 is an exploded side perspective view of the apparatus of FIG. 7;

FIG. 9 is an exploded three-dimensional view of the apparatus of FIG. 7;

FIG. 10 is an exploded three-dimensional view of a fourth embodiment of an apparatus for measuring soil respiration in soil;

FIG. 11 is an exploded side perspective view of the apparatus of FIG. 10;

FIG. 12 is an exploded three-dimensional view of a fifth embodiment of an apparatus for measuring soil respiration in soil;

FIG. 13 is an exploded side perspective view of the apparatus of FIG. 12;

FIG. 14 is a front perspective view of the apparatus of FIG. 12;

FIG. 15 is a block diagram of an embodiment of a system for measuring soil respiration in soil;

FIG. 16 is a graph of the data obtained in a field plot showing daily oscillation in soil respiration using an embodiment of the apparatus that includes a carrier gas;

FIG. 17 is a graph of the data obtained in a field plot showing daily oscillation in soil respiration using an embodiment of the apparatus that does not include a carrier gas;

FIG. 18 is a graph of the permeability of varying thicknesses of membranes exposed to known concentrations of gaseous CO₂;

FIG. 19 is graphs of the CO₂ permeability of varying thicknesses of membranes (A—1 mm; B—0.5 mm; C—0.2 mm; D-control without membrane) exposed to known concentrations of gaseous CO₂;

FIG. 20 is a graph of the gaseous CO₂ production in soil during water and fertilizer application, using various thicknesses of membrane-enclosed sensors (1 mm, 0.5 mm and 0.2 mm thick membranes) as well as an unenclosed control sensor;

FIG. 21 is a graph of the response of sensors enclosed with CO2 permeable membranes of various thicknesses (A—1 mm; B—0.5 mm; C—0.2 mm; D—control without membrane) to water and fertilization while buried in soil;

FIG. 22 is a graph of the permeability of varying thicknesses of membranes exposed to known concentrations of gaseous CO₂ with active pumping of atmospheric air that acts as a carrier gas into and out of the sensing systems;

FIG. 23 is a graph of the CO₂ permeability of varying thicknesses of membranes (A—1 mm; B—0.5 mm; C—0.2 mm; D—control without membrane) exposed to known concentrations of gaseous CO₂ with active air pumping;

FIG. 24 is a graph of the gaseous CO₂ production in soil during water and fertilizer application, using various thicknesses of membrane-enclosed sensors (1 mm, 0.5 mm and 0.2 mm thick membranes) as well as an unenclosed control sensor, with active air pumping;

FIG. 25 is a graph of the responsiveness to CO₂ levels of various thickness (A—1 mm; B—0.5 mm; C—0.2 mm; D—control without membrane) of membranes in untreated soil during water and fertilization with active pumping;

FIG. 26 is a graph of the effect of living plants on CO₂ production in soil as monitored by a passive system;

FIG. 27 shows three graphs of the effects of living plants on CO₂ production in soil as detected by the system;

FIG. 28 is a graph of the effects of sterilized, sterilized with microbial amendment and unsterilized soil on CO₂ detected; and

FIG. 29 shows three graphs of the effects of sterilized, sterilized with microbial amendment and unsterilized soil on CO₂ production.

### DETAILED DESCRIPTION WITH REFERENCE TO THE DRAWINGS

In an embodiment of the invention there is provided an apparatus and system for measuring the concentration of a gas, such as carbon dioxide, within soil. The system and apparatus enable the monitoring, in real time, of soil respiration rates (such as microbial, microfauna and plant metabolic activity) and other physical parameters (such as temperature, soil moisture levels, etc.) through the implementation of various Internet of Things (IoT) sensors and microcontrollers. Soil respiration rates may be determined by measuring the concentration of gasses within the soil such as carbon dioxide (CO₂). Other gasses in the soil such as oxygen (O₂), nitrogen (N₂), hydrogen sulfide (H₂S), and methane (CH₄) may also be measured.

The apparatus comprises a tubular housing and a gas sensor. The term “tubular” refers to the broad definition of “having the shape of a tube” and refers to something that is generally long and hollow. The term “tubular” does not limit the shape of the housing to being round or circular. The housing is configured to extend beneath the soil surface and allow gasses in the soil to pass into the housing. The housing is in fluid communication with the sensor to allow the gasses to reach the gas sensor where measurements, such as carbon dioxide levels, may be taken.

The tubular housing includes an outer protective sleeve or soil guard with perforations or holes therein. The sleeve may act as a barrier to limit soil and debris from infiltrating the interior of the housing and damaging any components housed within the housing. The housing further includes a support structure that is gas permeable. The support structure is surrounded by the outer protective sleeve and defines an inner chamber. The support structure carries a gas permeable membrane, such as a carbon dioxide permeable silicone membrane, configured to prevent ingress of soil or liquid into the inner chamber but allowing gas in the soil to diffuse into the inner chamber. The gas sensor is in communication with the inner chamber to measure a concentration of the gas in the inner chamber.

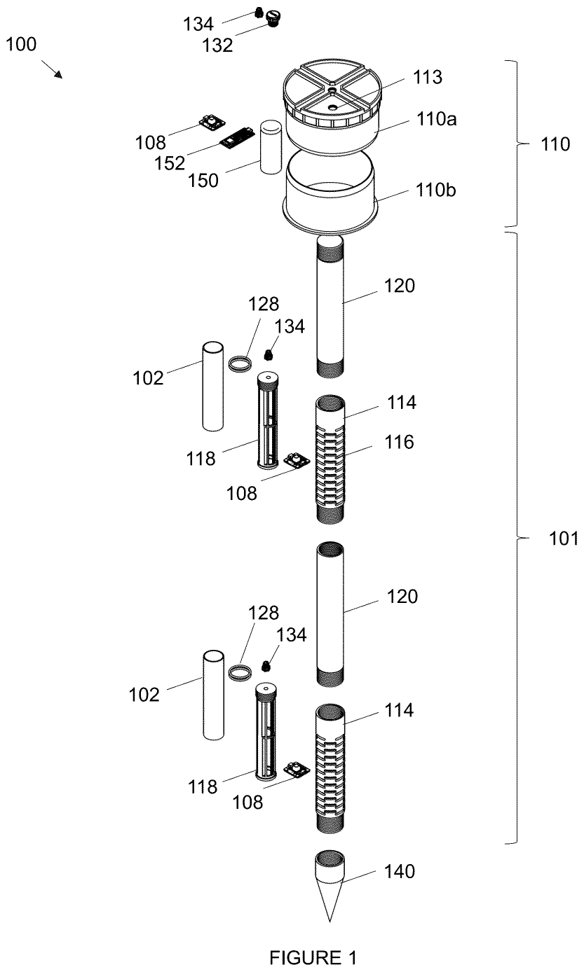

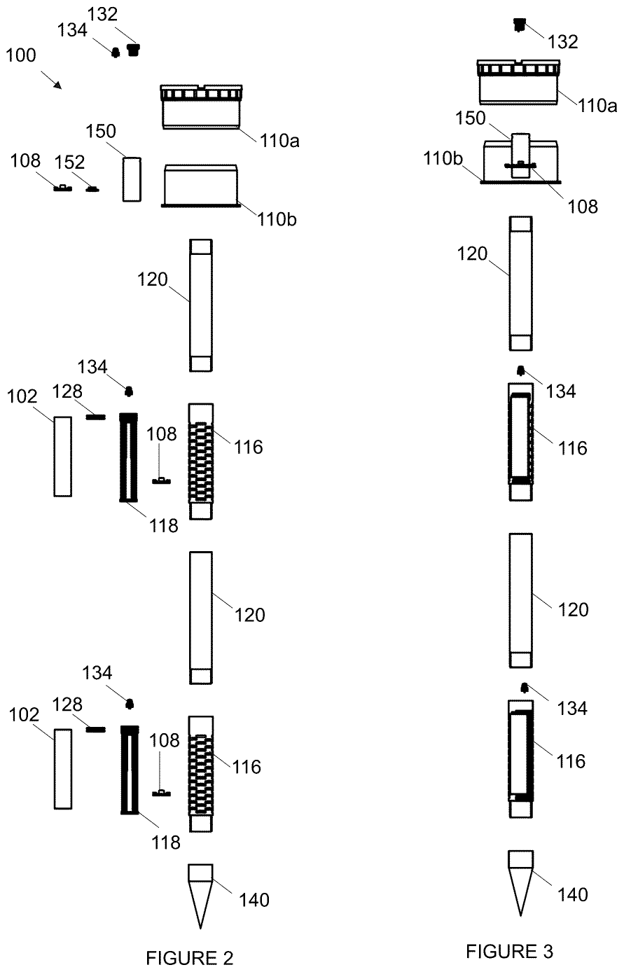

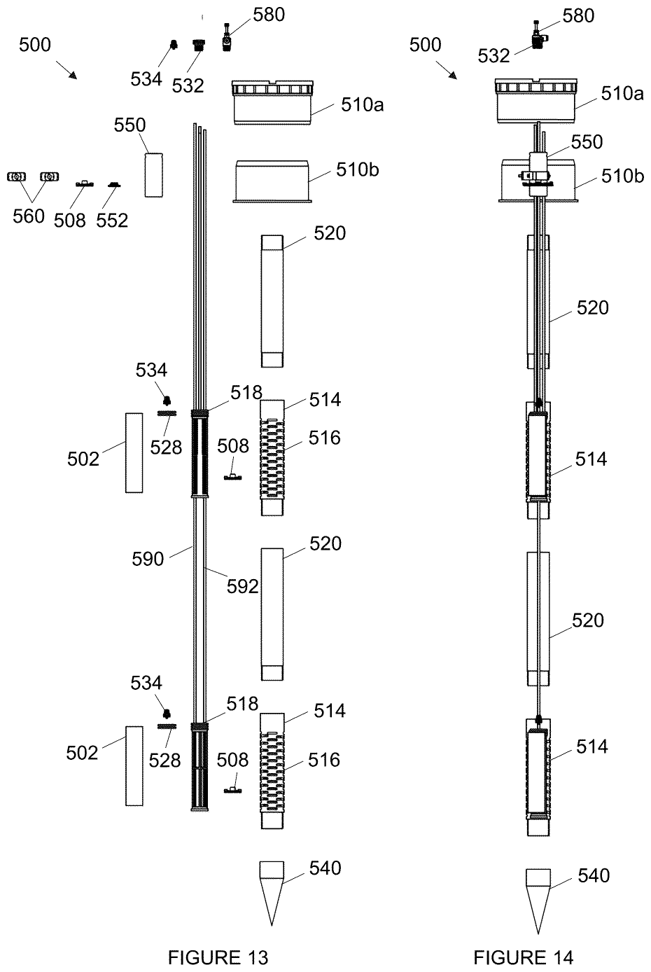

FIGS. 1 to 15 show different embodiments of the apparatus. The apparatus can take in-situ measurements of gasses in soil via two routes, namely actively (mechanically assisted) or passively (no mechanical assistance).

FIGS. 1 to 3 show an embodiment where the “passive” route may be followed. As shown in FIG. 1, the apparatus (100) has a tubular housing (101) configured to extend beneath the soil. The apparatus may also include a spiked end (140) for easier insertion into soil. The housing includes an outer protective sleeve (114) or soil guard with perforations (116). The plurality of openings or perforations (116) in the sleeve (114) provide fluid communication between the soil and the internal cavity of the sleeve.

The sleeve (114) receives a support structure (118) that defines an inner chamber. The support structure (118) carries a gas permeable membrane (102) configured to prevent ingress of soil or liquid into the inner chamber. The apparatus (100) further comprises a gas sensor (108). The sensor (108) is in communication with the inner chamber to measure a concentration of the gas in the inner chamber. As shown in FIG. 1 to 3, the apparatus may have more than one gas sensor (108).

The length of the housing may be adjusted by extensions (120). These extensions (120) may have a predetermined length for monitoring gasses in a soil zone of interest. For example, if a soil zone of interest is deeper than the housing is long, an extension may be added to the housing to lengthen it so that the section of the housing containing the gas permeable membrane is in the soil zone of interest. The extension (120) may have an outer wall that is not gas permeable.

The apparatus (100) may have a compartment (110) configured to receive the sensor (108). The compartment may be adapted to contain the sensor and may further be configured to provide protection to the components within the compartment against bumps, water, dust, etc. The compartment may be insulated. Additional components of the apparatus may also be stored in the compartment, for example batteries (150), microcontrollers (152), telemetry models and/or other sensors. The gas sensor (108) may be secured within the housing (101) or may be stored in the compartment (110), or both. The sensor compartment (110) may have a top (110a) and bottom (110b) section. The two sections may be configured to be releasably secured together. The compartment may be sealed by a gas tight seal.

An extension (120) may space apart the compartment (110) from the inner chamber by a predetermined distance selected for monitoring gasses in a soil zone of interest. As shown, the apparatus may take measurements of more than one soil zone of interest by having two or more gas sensors and two or more protective sleeves interspersed with two or more extensions (120). The components may be connected to each other via screw threading. Nitrile O-rings (128) may be used to form a seal at connection points. The extension(s) (120) is in fluid communication with the inner chamber and configured to allow gas diffusion from the inner chamber to the compartment (110).

An outlet (113) may be located at the top of the compartment (110). This outlet (113) may include a corrosion resistant air flow valve (132) and a vent to provide an exit for the gasses within the compartment and a weatherproof data and power connector (134). Weatherproof data and power connectors may also be located within the housing of the apparatus.

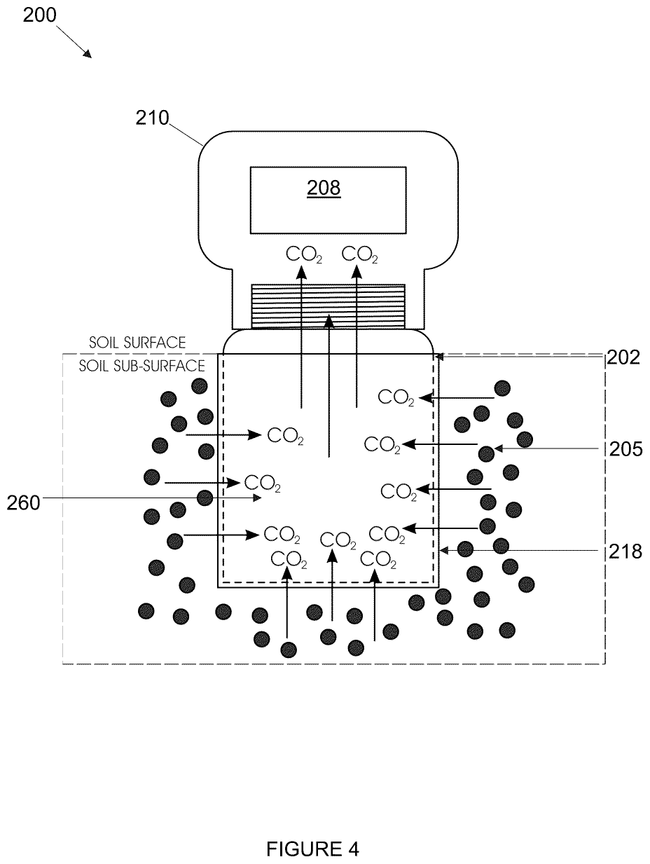

FIGS. 4 to 6 show another embodiment where the “passive” route (i.e., not requiring a pump and carrier gas) may be followed. The arrows indicate gas movement through the system. The measurements for this embodiment are applicable to the root zone of the soil and to assessing overall flux from soil to atmosphere. FIG. 4 shows a basic representation of the flow of gasses within such a system. The apparatus (200) has a silicone membrane (202) that forms an inner chamber or air-filled cavity (260). A perforated support structure (218) may be provided to support the membrane. CO2 (205) and other gasses migrate from the soil through the membrane (202) and into the inner chamber or cavity (260). The CO₂ migrates up the inner chamber (260) to the compartment (210) where a first sensor (208) may be stored.

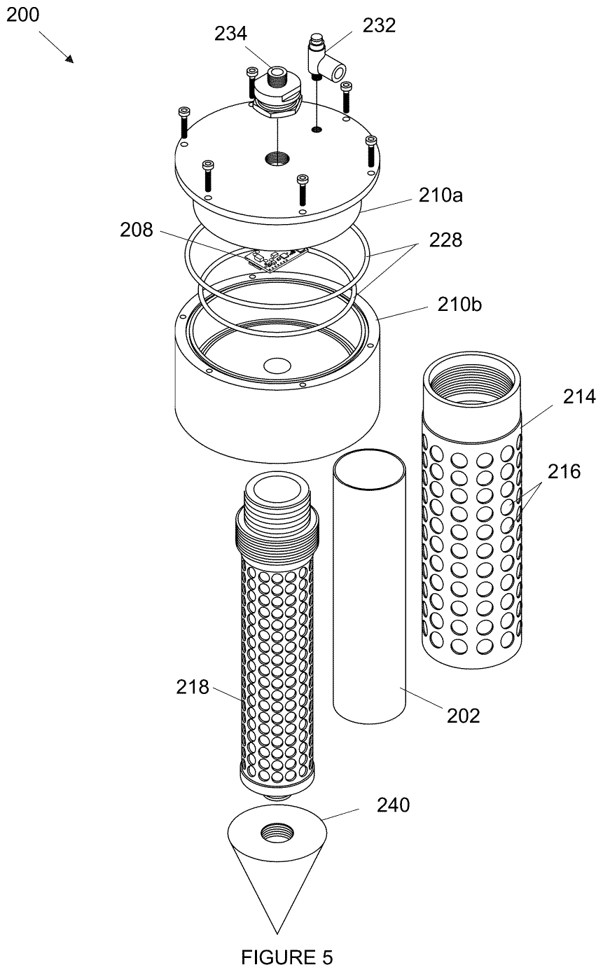

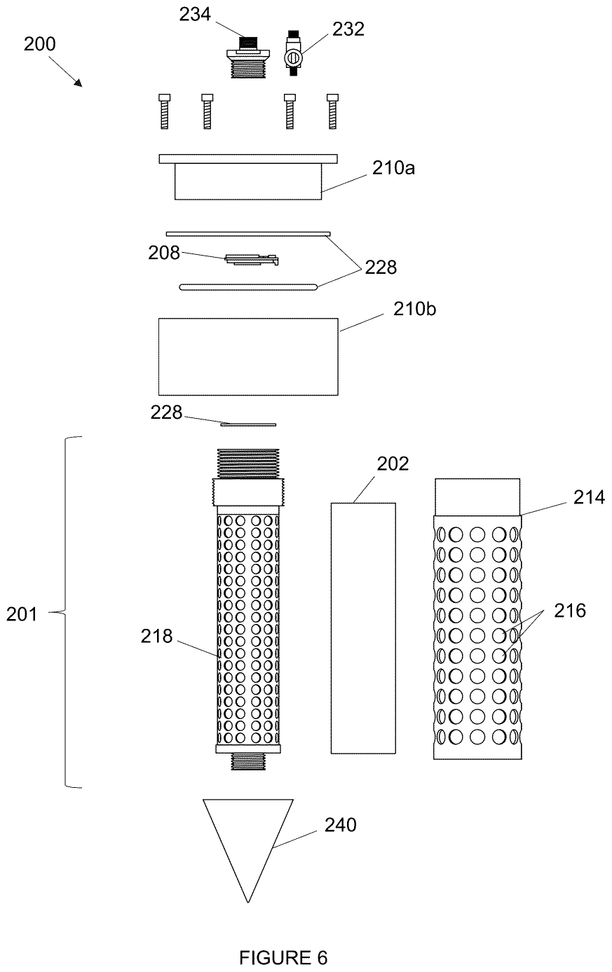

FIGS. 5 and 6 provide a more detailed embodiment of the apparatus in FIG. 4. While FIG. 4 shows the apparatus with a generally flat base that allows for gas exchange from the bottom, the apparatus (200) may have a spiked end (240) as shown in FIGS. 5 and 6 to allow for easier insertion into a top layer of soil. The spiked end may also allow for gas exchange to occur. The apparatus (200) comprises a housing (201) and a sensor (208). A perforated guard or protective sleeve (214) with holes (216) may be provided for housing the permeable silicone membrane (202) that forms an inner chamber. A perforated support structure (218) may also be provided. The silicone membrane (202) is attached to the outside of the perforated support (218) to create the inner chamber. The protective sleeve (214) encloses both the membrane (202) and the support (218) to provide protection from soil debris or particles.

As with the previous embodiment, a compartment (210) with a top (210a) and bottom (210b) section may be provided to house the sensor (208). Nitrile O-rings (228) may be used to form a seal at connection points. An outlet may be located at the top of the compartment (210). This outlet may include a corrosion resistant air flow valve (232) and a weatherproof data and power connector (234).

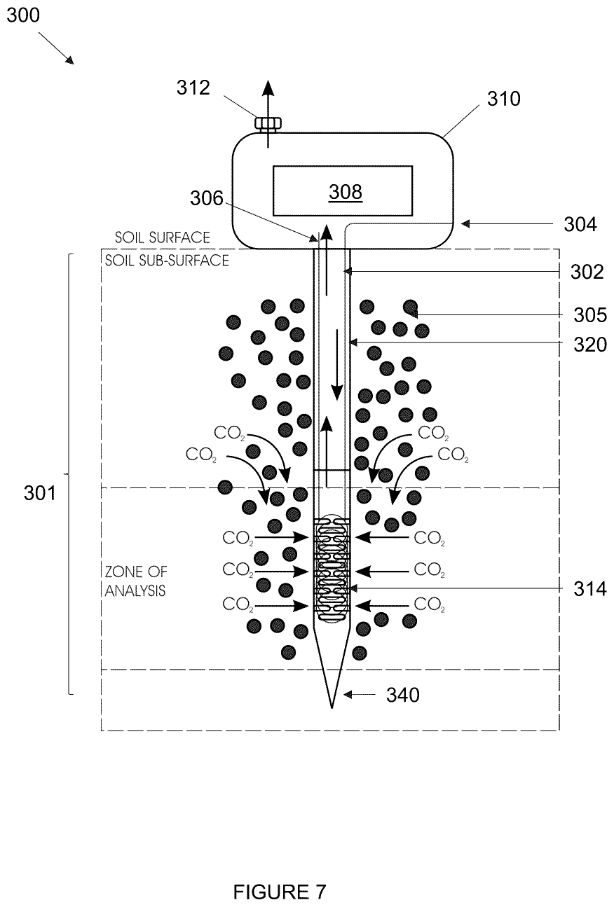

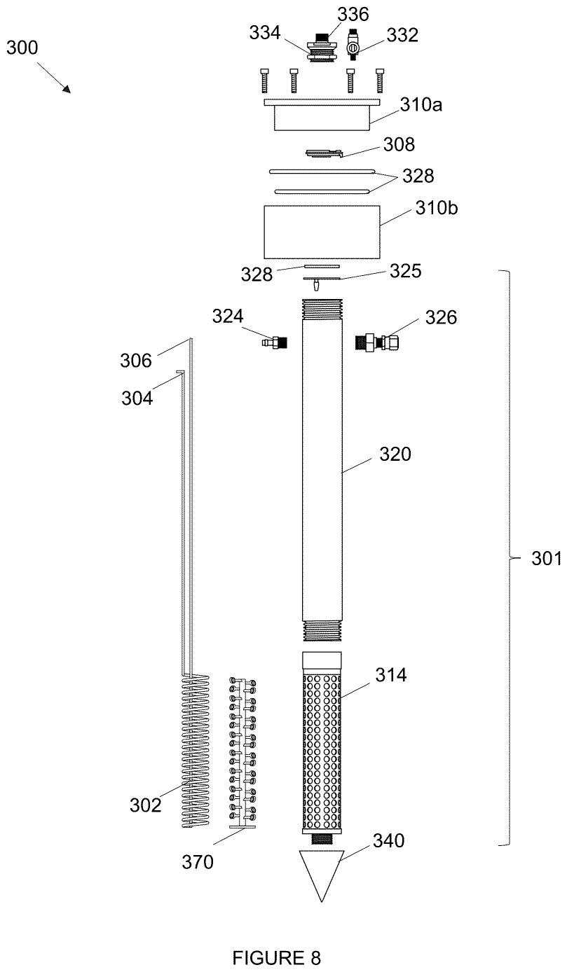

Another embodiment of the apparatus is shown in FIGS. 7 to 9. This embodiment has a similar design to what is shown in FIGS. 4 to 6 but includes a pump and carrier gas and therefore follows the “active” route. FIG. 7 shows a basic representation of the flow of gasses within this embodiment. The apparatus (300) has an elongated housing (301), shown in FIG. 8, that is divided up into an extension or intermediate section (320) that is impermeable and a protective sleeve (314) that is permeable. The spiked end (340) may form part of the housing or may be a separate component. A gas permeable membrane, such as silicone tubing (302) is enclosed within the housing (301). The gasses (305) in the soil may migrate from the soil through the permeable sleeve (314) and into the tubing (302). This apparatus (300) may include a pump (not shown) to allow for the pulling of atmospheric air through the system from the first end (304) of the tube to the second end (306) of the tube that terminates inside a compartment (310) that encloses a first sensor (308). Additional sensors may also be provided. The compartment (310) may have a vent (312) to allow the gasses to escape. The vent may allow for the controlled release of gasses from the system. The apparatus may extend to various depths, for example if it extends to a depth of about 85 cm the zone where gas transfer to the inner chamber or tubing occurs is at a depth of about between 50 cm to 85 cm.

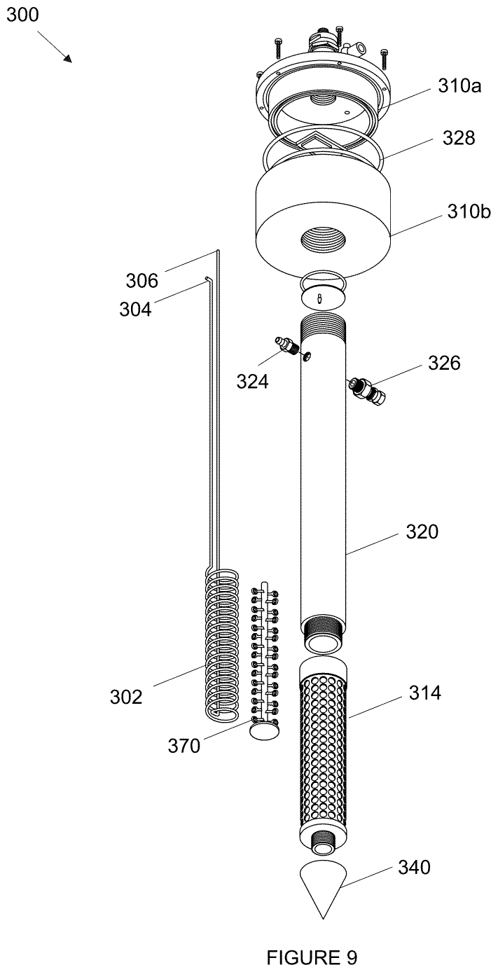

FIGS. 8 and 9 provide a more detailed embodiment of the apparatus in FIG. 7. The apparatus (300) may have a spiked end (340) to allow for easier insertion into soil. The elongated body or housing (301) of the apparatus may be divided into an extension or intermediate section (320) that is impermeable and a protective sleeve or guard (314) that is permeable. The sleeve (314) encloses a permeable silicone tube (302) that forms an inner chamber. The tube (302) may be coiled, and a support frame (370) may assist in maintaining the coiled configuration of the tube. As explained above, the tube has a first end (304) and a second end (306). The first end (304) is where a carrier gas enters the tube, and the second end (306) is where the carrier gas and other collected soil gasses (such as CO₂) exit the tube. The extension (320) may have a first barbed connector (324) configured to be located on a section of the housing (301) that extends above the soil surface. This first barbed connector (324) may receive the first end (304) of the tube. An air valve (326) may also be provided on the extension (320) to allow for unclogging of the protective sleeve (314) with compressed air if required. The components may be connected to each other via screw threading. A second barbed connector (325) may be provided to receive the second end (306) of the tube within the compartment (310).

As with the previous embodiment, a compartment with a top (310a) and bottom (310b) section may be provided to house the sensor (308). Nitrile O-rings (328) may be used to form a seal at connection points. An outlet may be located at the top of the compartment. This outlet may include a corrosion resistant air flow valve (332) and a weatherproof data and power connector (334). The outlet may also include a connector (336) in communication with a system controller.

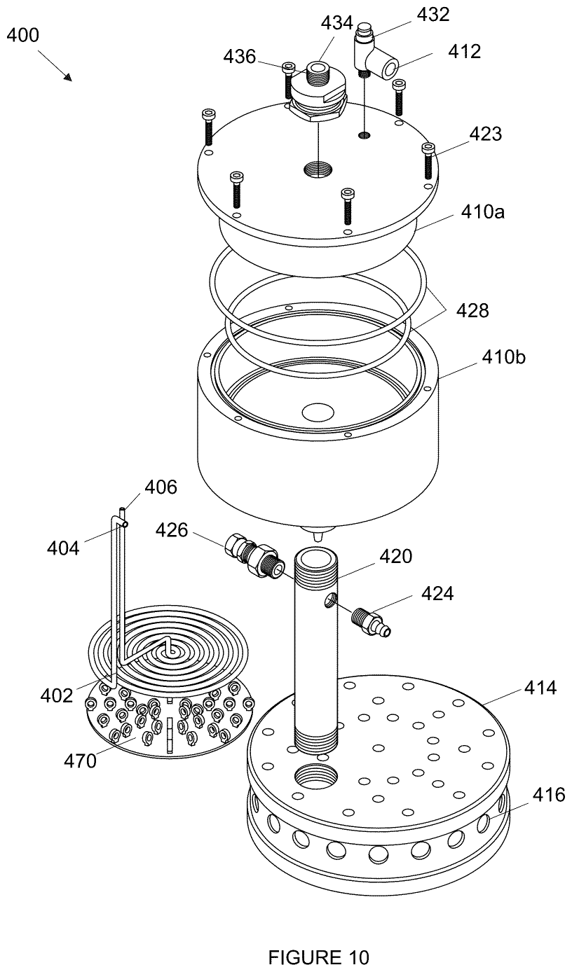

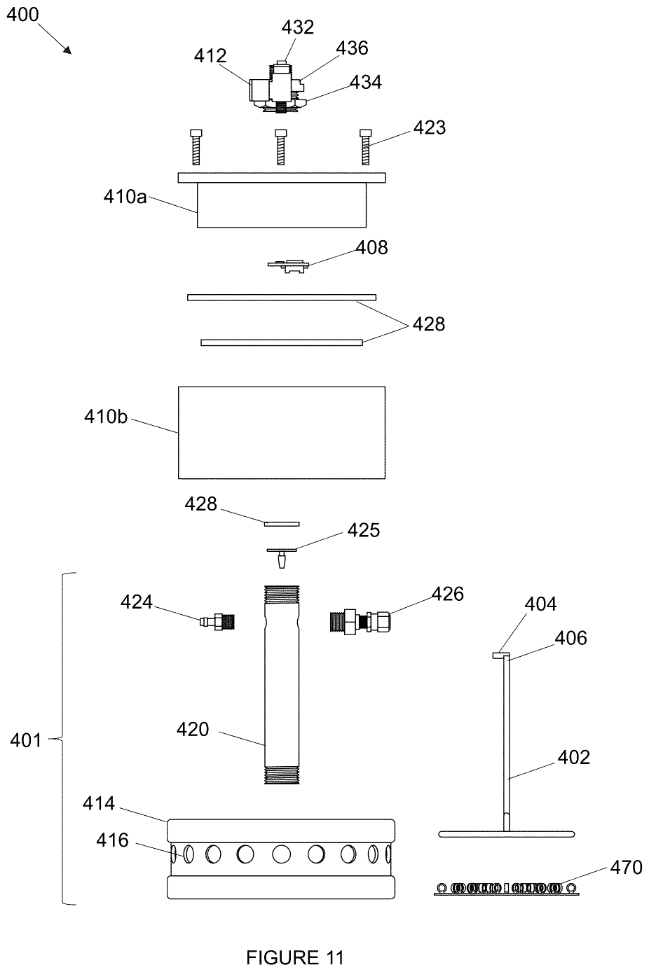

FIGS. 10 and 11 show another embodiment of the apparatus that operates with a pump and carrier gas. In this embodiment, the apparatus (400) has a housing (401) with a perforated sleeve or soil guard (414) which receives the coiled gas permeable membrane (402). The plurality of openings or perforations (416) in the soil guard provide fluid communication between the soil and the tubing thereby allowing migration of soil gasses from the soil into the housing and up to the sensor (408).

A tubing support frame (470) is provided to assist in keeping the tube in a coiled configuration. As explained above, the tube has a first end (404) and a second end (406). The first end (404) is where a carrier gas enters the tube (402), and the second end (406) is where the carrier gas and other collected soil gasses (such as CO₂) exit the tube. The apparatus may have an extension or intermediate section, such as a pipe (420), that houses the tube (402) as it extends from the perforated guard (414) to a sensor compartment (410) that may have a top (410a) and bottom (410b) section secured together with screws (423). The pipe (420) may have a first barbed connector (424) configured to be located on a section of the pipe (420) that extends above the soil surface. This first barbed connector (424) may receive the first end (404) of the tube. An air valve (426) may also be provided on the pipe (420) to allow for unclogging of the perforated guard with compressed air if required. The components may be connected to each other via screw threading. A second barbed connector (425) may be provided to receive the second end (406) of the tube within the sensor compartment (410). Nitrile O-rings (428) may be used to form a seal at connection points. A first sensor (408) for measuring CO₂ may be housed within the sensor compartment (410). An outlet may be located at the top of the sensor compartment (410). This outlet may include a corrosion resistant air flow valve (432) and a vent (412) to provide an exit for the gasses within the compartment and a weatherproof data and power connector (434). The outlet may also include a connector (436) in communication with a system controller.

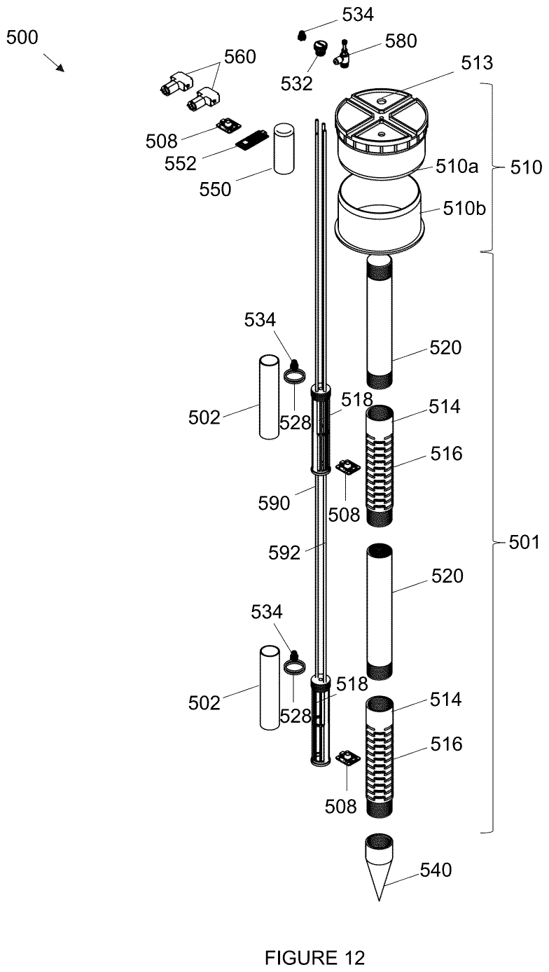

FIGS. 12 to 14 show, like FIGS. 7 to 11, an embodiment of the apparatus that operates with a pump and carrier gas. The apparatus (500) has a tubular housing (501) configured to extend beneath the soil with a spiked end (540) for easier insertion into soil. The housing includes an outer protective sleeve (514) with perforations (516).

The sleeve (514) surrounds a support structure (518) that defines an inner chamber. A gas permeable membrane (502) is supported by the support structure (518). The apparatus (500) further comprises one or more gas sensors (508). The sensor (508) is in communication with the inner chamber to measure a concentration of the gas in the inner chamber.

The apparatus (500) may have a compartment (510) to contain the sensor (508). Additional components of the apparatus may also be stored here, for example batteries (550), microcontrollers (552), telemetry models and/or other sensors. The gas sensor (508) may be stored within the housing (501) or in the compartment (510), or both. The sensor compartment (510) may have a top (510a) and bottom (510b) section. The two sections may be configured to be releasably secured together. The seal between the two sections may be a gas tight seal.

An extension (520) may space apart the compartment (510) from the inner chamber by a predetermined distance selected for monitoring gasses in a soil zone of interest. As shown, the apparatus may take measurements of more than one soil zone of interest by having two or more gas sensors and two or more protective sleeves interspersed with two or more extensions (520). The components may be connected to each other via screw threading. Nitrile O-rings (528) may be used to form a seal at connection points.

The compartment (510) may have an outlet (513). This outlet (513) may include a corrosion resistant air flow valve (532) and a weatherproof air flow control valve (580) to provide an exit for the gasses within the compartment and a weatherproof data and power connector (534). Weatherproof data and power connectors may also be located within the housing of the apparatus.

The apparatus (500) may also include gas permeable tubing (590, 592) configured to connect to an air pump (560) at one end. The tubing (590, 592) may be connected at the other end to the support structure (518). The air pump (560) pumps a carrier gas through the tubing and into the inner chamber. The carrier gas may increase the rate at which the gas from the soil reaches the sensor. One of the tubes (592) allows for air to flow out of the apparatus and the other tube (590) allows air to flow in. The air pressure inside the inner chamber may be controlled by managing the flow of air in and out of it.

Each additional apparatus may have its own tubing for air flow in and out of the system and may be connected to a separate pump. This may allow for multiple depths to be monitored at the same time.

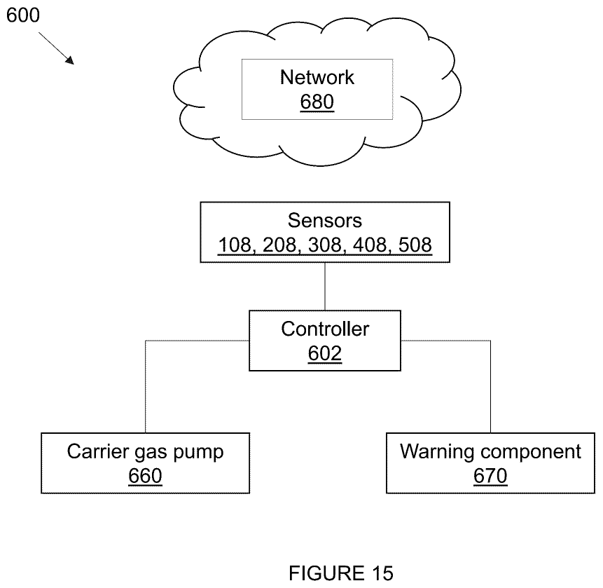

FIG. 15 shows a block diagram of an embodiment of a system (600) for measuring soil respiration. The system (600) includes a controller (602) configured to be in data communication with the sensors (108, 208, 308, 408, 508) of the apparatus(es) to continuously monitor the soil respiration measured. The controller may also be in data communication with additional sensors of the apparatus(es). The controller (602) may further be in data communication with a carrier gas pump (660) and a warning component (670). The sensors (108, 208, 308, 408, 508) may be wirelessly connected to a network (680) to allow for in-situ, real-time measurements.

The sensors may be “Internet of Things” or “IoT” enabled sensors. The sensors may be IoT enabled in that they may, for example be configured (e.g., through processing ability, software and other technologies) to connect to and exchange data with other devices and systems over the Internet or other communications networks. Through these types of data communications, real-time monitoring is possible.

In some embodiments, the individual sensors may be IoT enabled while in other embodiments the apparatus itself may provide the IoT functionality (e.g., through a controller and communication module which interface with the sensors and other devices and/or communication networks). The sensors may therefore be wirelessly connected to a network to allow for in-situ, real-time measurements. The apparatus and system therefore do not need to be removed from its location to further process the retrieved data thereby allowing continuous measurement. “Measuring flux” generally refers to the escape of gas from the soil surface to the atmosphere. The current apparatus and system are not restricted to flux measurements (soil-to-atmosphere release) and can link such measurements with in-situ measurements across the soil profile or in the root zone. The soil profile refers to the different vertical layers (termed horizons or zones) with the top zones containing most nutrients and receiving most moisture in dry areas. While roots can extend very deep into the soil, the primary root zone is typically in the top 2 horizons/zones generally referred to as topsoil, and therefore where most gas exchange occur. For that reason, most measurements of gas levels in soil are focussed on the topsoil.

The carbon dioxide levels measured generally represent carbon dioxide production or respiration rates of living organisms, such as microbes and plants, which may be present in the soil. Changes in the carbon dioxide levels as measured by the carbon dioxide sensor indicate a relative amount of carbon dioxide diffusing through the carbon dioxide permeable membrane. The coiled arrangement of the permeable membrane provides a larger surface area for collecting gasses.

The system may include a warning component configured to be in communication with the controller and emit a warning signal if the parameters measured by the apparatus and monitored by the controller reach a selected threshold level. The system can, for example, provide an early warning of waterlogging.

The controller may also be configured to be in communication with a pump that moves the carrier gas through the apparatus to control the flow rate of the carrier gas. The movement of the carrier gas through the system may be one-directional flow from the atmosphere through the system and back out to the atmosphere. As a result of these features, the system can be used as a real-time monitoring system that detects soil respiration in situ and under continuous flow.

The apparatus may include additional sensors/probes that, for example, measure the temperature, soil moisture and pH of the soil to continuously monitor these parameters and warn users of unfavourable conditions that will lead to plant stress.

Many parts of the apparatus, and in particular the housing, and the various parts of the sensor chamber may be made from plastic, stainless steel or a combination of both.

As is evident from the different embodiments shown in FIGS. 1 to 15, the apparatus and system may use a pump and carrier gas to transport the gasses in the soil to the sensor, but it is also possible to have a passive system where a pump and carrier gas is not required.

The extension or intermediate section located between the sensor compartment and protective sleeve has a length configured for measuring a soil zone of interest by spacing the perforated guard/protective sleeve away from the compartment by a distance that permits location of the perforated guard in the soil zone of interest and the compartment above the soil surface. The apparatus may generally extend between about 10 cm to 100 cm into the soil from the soil surface. While measurements are generally taken of the topsoil, there may be applications where measurements of deeper soil zones, such as the plough soil (dense zone), may be required. These deeper soil zones may develop deeper than 1 meter below the soil surface and may require the apparatus to extend further than 1 meter into the soil.

The perforations of the guard/sleeve and the support structure may be circular or rectangular or another functional shape. The perforations of the guard are intended to allow gas transfer from the soil to the inner chamber while protecting the inner components of the housing and limiting soil and debris from entering the housing. The perforations of the support structure are intended to allow a large surface area for the membrane to optimise gas transfer from the soil to the inner chamber while limiting ingress of debris and liquid into the inner chamber. The framework of the support structure may be configured to limit damage to the membrane, i.e., the framework may have smooth edges so that when the membrane is secured to it, the chances of the membrane being punctured is reduced. The membrane may cover the support structure or may be secured to the inside of the support structure.

In addition to using a sensor (such as a NDIR CO₂ sensor) for monitoring carbon dioxide in the soil, the apparatus may also include sensors for other gasses (NH₄, O₂ etc.) found in soil. The measurement of physicochemical parameters (i.e., pH, temperature, etc.) may also be carried out with the apparatus.

An example of an application of the apparatus and system is in the monitoring of waterlogging. Waterlogging is the saturation of soil with water that displaces air in soil and leads to oxygen deficiency, more alkaline conditions, and as a result, significantly reduced plant growth. In short, waterlogging occurs when air pockets in the soil are filled with water and the plants “drown”. Waterlogging can be prevented through optimized irrigation in terms of the frequency, amount, and optimization of plant growth. The apparatus and system may be used to enable precision irrigation by measuring and recording carbon dioxide levels, dissolved oxygen levels and other relevant parameters in real-time to prevent exceeding of the soil's water holding capacity.

The current apparatus and system may allow for simple repair and replacement of components due to its modular design. A cheaper apparatus may allow for small scale use of the apparatus. With a shortage of agricultural extension officers to assist upcoming farmers with research and sustainable farming practices, the system has potential to bring technology to small-scale farming, where quality control is essential to enable the farmers to penetrate the formal market (i.e., chain stores) with their produce.

By using the current apparatus and system to monitor the soil respiration values, a user may get an indication of the soil respiration rate which may indicate a lack or excess of fertilizer. The potential for notable savings in the context of rapidly rising production cost due to fertilizer and energy cost is equally important to small and large commercial farmers, and eventually, consumers. Measuring microbial metabolism (CO₂ production) may therefore assist in finding the balance between irrigation and bio-stimulant application.

The current apparatus and system allow for real-time recording of microbial/microfauna respiration that reflects soil health, or activity of microbes in soil systems. The system measures respired CO₂ and other gasses of interest in the soil that cross a permeable membrane and are collected for immediate analysis by a carbon dioxide sensor and other appropriate gas sensors as required. The gasses of interest in the soil may passively cross the permeable membrane and diffuse to the sensor or may be pumped to the sensor via a pumping system and a carrier gas. Other sensors may include oxygen and/or ammonia/ammonium sensors. The sensors may be mounted on a generally vertical support to, in use, ensure adequate gas flow around the sensor. The carbon dioxide sensor may be a nondispersive infrared sensor (NDIR) configured to measure carbon dioxide concentrations. It typically has a dynamic range and in effect measures the flux of carbon dioxide across the carbon dioxide permeable membrane. The NDIR sensor may also be configured to measure the temperature and humidity together with the changing carbon dioxide levels in the carbon dioxide measurement chamber.

### EXPERIMENTAL DATA

### Trial 1

Proof-of-concept experiments for both the active (with a pump and carrier gas) and passive (without a pump and carrier gas) embodiments of the apparatus were conducted in a field plot (residential garden). Both systems made use of NDIR CO₂ sensors to measure the CO₂ concentration contained in the air and in the soil. The soil data obtained from the sensor was logged via a microcontroller. Slight differences exist between the active and passive embodiments with respect to the silicone membrane orientation and form.

The active and passive apparatuses were positioned within close proximity in the soil (within 10 cm of each other). After both apparatuses were placed in the test plot, the area was left undisturbed for 2 weeks prior to commencement of data collection (to allow soil to re-equilibrate to environmental conditions). Data, including CO₂, humidity, and temperature, were measured at 1-minute intervals over the entire experimental period.

The field plot was automatically irrigated daily for periods of 15 minutes and subjected to prevailing environmental conditions. Organic, liquid fertilizer was applied according to manufacturer's recommendations after±175 h, followed by resumption of normal irrigation frequency.

Active embodiment with pump and carrier gas:

The active embodiment of the apparatus made use of 2 meters of silicone tubing (1.0 mm wall thickness) coiled/spiralled in the upper part of the soil (soil horizons A: topsoil/surface soil). The insertion of silicone tubing was achieved by excavating a field plot measuring 30 cm×15 cm to a depth of approximately 15-20 cm. The spiralled/coiled silicone tube was then inserted into the hole and backfilled.

The open ends of the silicone tubing were connected via barbed connectors with one end being terminated to a port connected to the outside air, while the other end of the tube was terminated near the CO₂ sensor. The mechanical movement of air through the system was achieved using a peristaltic pump that connected in such a way to allow for the pulling of atmospheric air through the system. The atmospheric air acted as a carrier gas although any suitable carrier gas may be used.

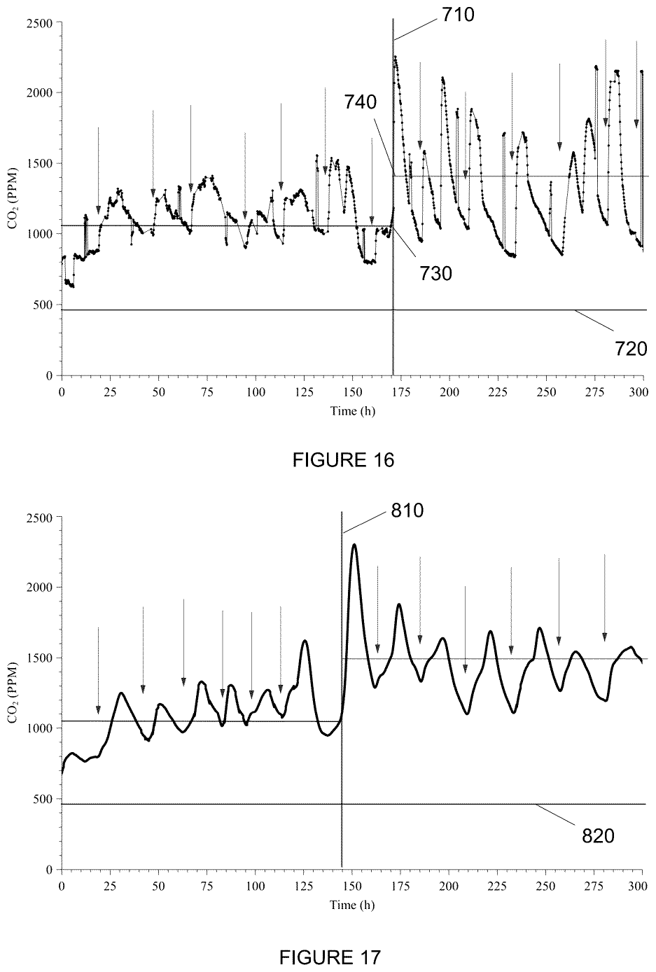

FIG. 16 shows the data obtained in the field plot using the active embodiment of the apparatus. The apparatus measured the response of soil respiration rates to daily irrigation (indicated with arrows) and after fertilizer application (710). The total average atmospheric CO₂ concentration (720) is 475 ppm. The average metabolic activity (730) in the soil between 0 -170 h was 1050 ppm and the new average increase in metabolic activity (740) in the soil between 1705-300 h (i.e., after fertilizer application) was 1500 ppm.

Passive embodiment without pump and carrier gas:

Whereas the active embodiment may require mechanical movement of air when the deeper soil zones are of interest, the passive embodiment does not rely on mechanical movement of air. Here, a silicone sheet/membrane (1.0 mm thickness, although experiments were also done with thicknesses varying between 0.25 mm to 1.5 mm in water to show predictable effect of sheet thickness i.e., inverse correlations between thickness and response time) is wrapped around a stainless-steel mesh/filter/strainer which provided the support structure and placed in the top 0 to 20 cm of the soil. The top of the steel filter had a threaded opening, while the bottom was solid. By wrapping the silicone around the filter, it formed a sealed cylinder that only permitted the transfer of CO₂ through the silicone membrane. The cylindrical silicone structure was then inserted into the upper part of the soil (soil horizons A: topsoil/surface soil). This was facilitated through the excavation of a cylindrical hole (10 cm in diameter and a depth of 15-20 cm). The silicone structure was inserted into the hole and backfilled.

Since this system is passive in nature due to the sensor being positioned right above the gas-exchange part, no mechanically aided movement of air was needed, and no peristaltic pump was required. Movement of the CO₂ from the soil environment to the sensor chamber is driven by diffusion.

FIG. 17 shows the data obtained in the field plot using the passive embodiment of the apparatus. The apparatus measured the response of soil respiration rates to daily irrigation (indicated with arrows) and after fertilizer application (810). The total average atmospheric CO₂ concentration (820) is 475 ppm. The average metabolic activity in the soil between 0-145 h was 1050 ppm and the new average increase in metabolic activity in the soil between 145-300 h (i.e., after fertilizer application) was 1500 ppm.

### Trial 2

### Evaluating the CO₂ Permeability of Varying Membrane Thicknesses (Passive System)

The aim of this experimental validation was to determine whether the thickness of the permeable membrane influenced the diffusion of known concentrations of gaseous CO₂.

Membranes with various thicknesses (0.2 mm, 0.5 mm and 1 mm) were wrapped around a cylindrical support structure containing a CO₂ sensor, to produce sealed sensing units. These units were placed in a sealed container with two ports, one for gas transport into, and the other for gas transport out of the container. This allowed for controlled airflow and maintenance of positive pressure within the container to limit the penetration of atmospheric gases. The sensors were thus isolated within the permeable membrane and protected from soil, water and ingress of other potentially damaging materials while still being permeable to gases. The control sensor was inserted into a cylindrical support structure without a membrane.

The sealed canister was flooded with a known concentration of gaseous CO₂ at a constant flow rate of 10 ml.l⁻¹. For the purposes of this experiment, 400-500 ppm was considered the ‘low’ concentration, and 2200 ppm as the ‘high’ concentration. The sensor CO₂ concentration readings were allowed to stabilize, and once the readings reached an approximate steady-state, the inflowing gas was changed from the low to high, or the high to low concentration. The sensor CO₂ concentration readings were allowed to stabilize.

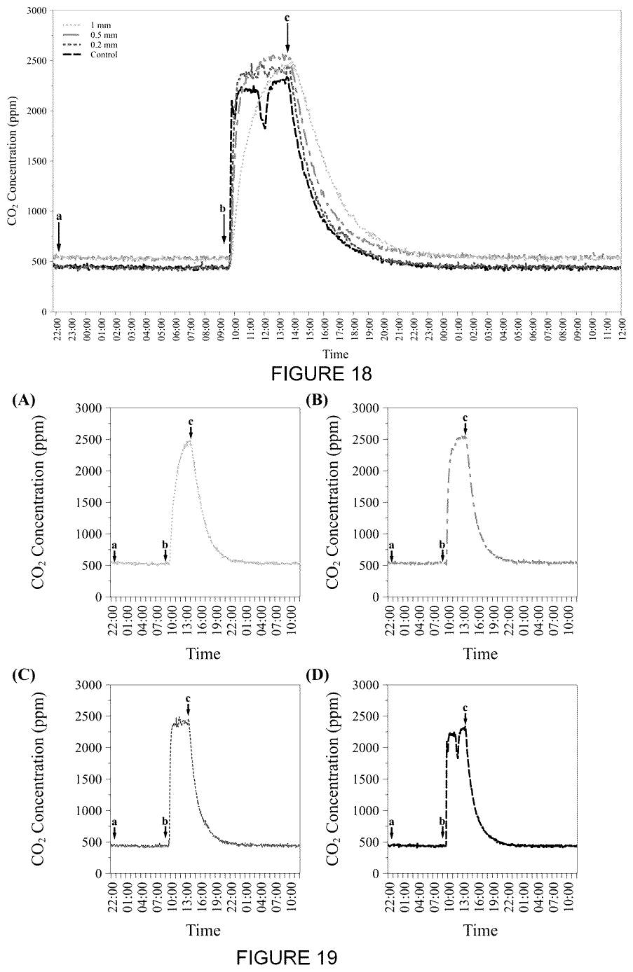

FIGS. 18 and 19 show the results. Arrow (a) illustrates the introduction of the low concentration of CO₂ (500 ppm) followed by the introduction of the high concentration of CO₂ (2200 ppm) as indicated with arrow (b). Once all of the sensors had stabilized at the higher concentration of CO₂, the low concentration (500 ppm, arrow c) was reintroduced, and the system was allowed to stabilize.

FIG. 18 shows the permeability of varying thicknesses of membranes exposed to known concentrations of gaseous CO₂. Three different thicknesses of membranes (0.2 mm, 0.5 mm and 1 mm) and a control sensor not enclosed in a membrane were assessed for permeability to known concentrations of CO₂ gas. Gas mixtures were introduced into the system in the following order (a) Nitrogen gas containing 500 ppm CO₂, (b) Nitrogen gas containing 2200 ppm CO₂ and (c) Nitrogen gas containing 500 ppm CO₂. All the membranes and exposed control sensor were sealed in a container, with only an inflowing and outflowing gas port to ensure a controlled gas flow (10 ml.min⁻¹) and positive pressure.

FIG. 19 shows CO₂ permeability of varying thicknesses of membranes exposed to known concentrations of gaseous CO₂. (A) 1 mm thick membrane, (B) 0.5 mm thick membrane, (C) 0.2 mm and (D) control sensor without a membrane. Varying gases of known CO₂ concentrations were flowed into the system in the following order (a) Nitrogen containing 500 ppm CO₂, (b) Nitrogen containing 2200 ppm CO₂, and (c) Nitrogen containing 500 ppm CO₂. All the membranes and control sensor were sealed in a container with only gas in and out ports to ensure a controlled air flow (10 ml.min⁻¹) and positive pressure in the container.

### Evaluating the Performance of Various Membrane Thicknesses When Buried in Soil

The aim of this experimental validation was to determine whether the thickness of the permeable membrane influenced the diffusion of gaseous CO₂ when buried in soil (in the absence of plants). Membranes with various thicknesses (0.2 mm, 0.5 mm and 1 mm) were wrapped around a cylindrical support structure containing a CO₂ sensor, to produce a sealed sensing unit. The unenclosed control sensor and enclosed sensing units (with a membrane and sensor) were placed in a plastic pot on a bed of potting soil (10-15 cm from the bottom of the pot) and buried in potting soil so that the tops of the sensing units were 10 cm below the soil surface.

The system was left undisturbed prior to the first watering event. During the watering event, water was equally distributed across the surface of the soil in a fine mist to ensure even distribution and prevent erosion that may potentially expose the top of the sensing units. After the watering event, the system (pot, soil, sensing unit etc.) was left undisturbed in a temperature-controlled environment for ±24 h prior to the second watering event. The second watering event followed the same procedure as the first, with the only difference being that the system was left undisturbed after this watering event for ±48 h. Thereafter, a commercial fertilizer was applied to the soil surface and followed by a third watering. The commercial fertilizer contained a high concentration of carbon (116 g/kg) and nitrogen (163 g/kg) and no phosphate or potassium (NPK+C=16:0:0+16) and was applied at the manufacturer's recommended dosage. Water was applied to the soil surface to dissolve the fertilizer and permeate into the soil. The system was the left undisturbed for 48 h prior to termination of experiment.

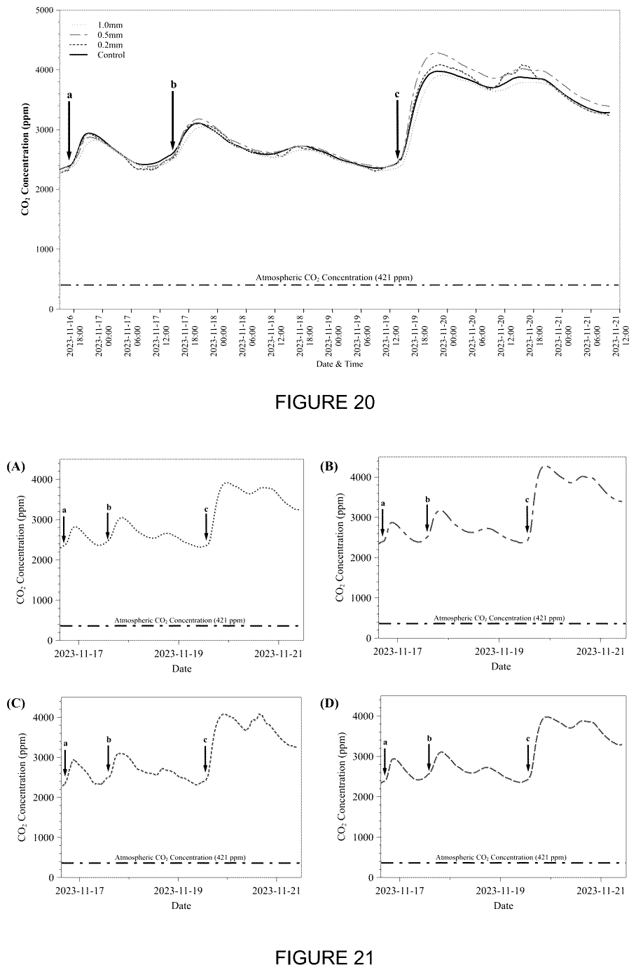

FIGS. 20 and 21 show the results of the experiment. Atmospheric concentrations of CO₂ is indicated by the horizontal dash-dotted line (±421 ppm in this case). An average CO₂ concentration of ±2200 ppm was measured for dry soil, and the addition of water (arrow a) caused an increase to 2800-2900 ppm, depending on membrane thickness. The second addition of water (arrow b) resulted in an increase in CO₂ concentration to 3028-3200 ppm depending on membrane thickness. The addition of the same volume of water in conjunction with a water-soluble fertilizer resulted in a rapid increase in detected CO 2 levels from ±2400 ppm to ±4100 ppm (arrow c) where after the CO₂ concentrations remained at these elevated levels for more than 30 h post-dosing. FIG. 21 shows the results of the different thickness of membranes (in mm): (A) 1 mm, (B) 0.5 mm, (C) 0.2 mm and (D) control Despite slight differences, all the membranes performed equally well as the control system.

### Evaluating the CO₂ Permeability of Varying Membrane Thicknesses (Active System)

The aim of this experimental validation was to determine whether the thickness of the permeable membrane influenced the detection of known concentrations of gaseous CO2 that is actively pumped through the system.

Membranes with various thicknesses (0.2 mm, 0.5 mm and 1 mm) were wrapped around a cylindrical support structure containing a CO₂ sensor, to produce sealed sensing units. The sensors were thus isolated within the permeable membrane and protected from soil, water and ingress of other potentially damaging materials while still being permeable to gases. The control sensor was inserted into a cylindrical support structure without a membrane. These units were placed in a sealed container with two ports, one for gas transport into, and the other for gas transport out of the container. This allowed for controlled flow of gas and maintenance of positive pressure within the container to limit the penetration of atmospheric gases. In addition, each individual sensing unit was modified by the addition of two ports; a gas inflow and gas outflow. Atmospheric air was actively pumped into each sensing unit via the gas inlet at a constant flow rate of 1 ml.min-⁻¹, allowed to mix with the gas that had diffused through the membrane from the inside of the sealed container, and the outflow of the resulting mixture via the gas outlet. This allowed for the continual movement and mixing of the gas enclosed within the sensing unit. The sealed container was flooded with a known concentration of CO₂ in N₂ (either ±400-500 ppm or 2200 ppm gaseous CO₂) at a constant flow rate of 10 ml.l⁻¹ until CO₂ readings stabilized. Once the readings reached an approximate steady-state, the gases were switched as described for the passive systems above.

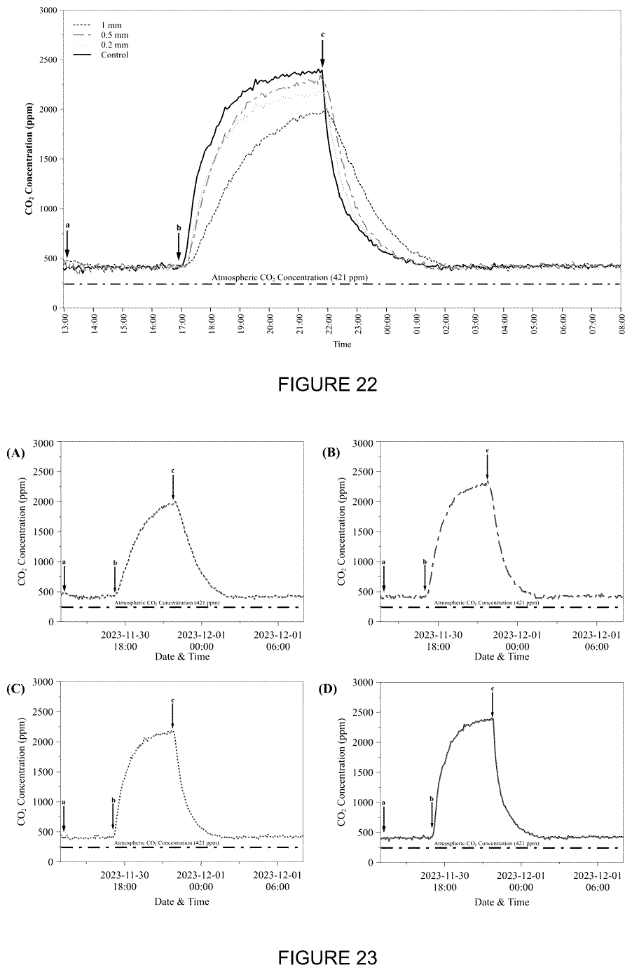

FIGS. 22 and 23 show the results. Arrow (a) illustrates the introduction of the low concentration of CO₂ (500 ppm) followed by the introduction of the higher concentration of CO₂ (2200 ppm) as indicated with arrow (b). Once the system all the sensors has stabilized at the lower concentration of CO₂, the low concentration of CO₂ (500 ppm, arrow (c)) was reintroduced with the system again allowed to stabilize.

FIG. 22 shows a graph of the permeability of varying thicknesses of membranes exposed to known concentrations of gaseous CO₂ with active pumping of atmospheric air into and out of the sensing systems. Three different thicknesses of membranes (1 mm-dotted line, 0.5 mm-dash-dotted line and 0.2 mm-dashed line) and a control sensor without any membrane were assessed for permeability to known concentrations of CO₂ gas. Gas mixtures were introduced into the sealed canister in the following order (a) Nitrogen containing 500 ppm CO₂, (b) Nitrogen gas containing 2200 ppm CO₂ and (c) Nitrogen containing 500 ppm CO₂. The flow of atmospheric air into the enclosed sensing units was maintained at 1 ml.min⁻¹. All the membranes and exposed control sensor were sealed in a container, with an inflowing and outflowing gas port to ensure a controlled air flow (10 ml. min⁻¹) and positive pressure.

FIG. 23 shows a graph of CO₂ permeability of varying thicknesses of membranes exposed to known concentrations of gaseous CO₂ with active air pumping. Three different thicknesses of membranes (A) 1 mm, (B) 0.5 mm, (C) 0.2 mm and (D) control sensor without any membrane were assessed to known concentrations of CO₂ gas. Gas mixtures were introduced to the system in the following order (a) Nitrogen containing 500 ppm CO₂, (b) Nitrogen gas containing 2200 ppm CO₂ and (c) Nitrogen containing 500 ppm CO₂. The circulation of air into the enclosed space of each sensing unit was maintained at 1ml.min⁻¹. All the sensing units and the control unit were sealed in a container with only gas in and out ports to ensure a controlled air flow (10 ml.min⁻¹) and positive pressure in the container.

### Evaluating the Performance of Various Membrane Thicknesses When in Buried in Soil

The aim of this experimental validation was to determine whether the thickness of the permeable membrane influenced the detection of gaseous CO₂ when buried in soil (in the absence of plants), while actively pumping atmospheric air into and out of the sensing units.

The same experimental set-up relating to the sealing of the CO2 sensors within a permeable membrane enclosure was followed as previously described. The sensing unit had two ports (gas in and out) that allowed for the exchange of the gas enclosed on the inside of the membrane. Atmospheric air was actively pumped through the gas inlet at a flow rate of 1 ml.min⁻¹ and allowed to mix with the gases that had diffused through the membrane from the surrounding soil. The sensor detected the concentration of CO₂ in the resulting gas mixture, which also flowed out via the gas outlet at the same flow rate.

The enclosed sensing units (membrane and sensor) were placed in a pot (10-15 cm from bottom of the pot on top of potting soil) and buried in potting soil so that the tops of the sensing units were 10 cm below the soil surface. The system was left undisturbed prior to the first watering event. During the watering event, water was equally distributed across the surface of the soil in a fine mist to ensure even distribution and prevent soil erosion that may have exposed the sensing units. After the watering event, the system (pot, soil, sensing unit etc.) was left undisturbed in a temperature-controlled environment for ±24 h prior to the second watering event. The second watering event followed the same procedure as the first with the only difference being that the system was left undisturbed after this watering event for ±18 h. A third watering event was applied after the addition of a commercial fertilizer to the surface of the soil in the pot. The fertilizer contained a high concentration of carbon (116 g/kg) and nitrogen (163 g/kg), but no phosphate or potassium (NPK+C=16:0:0+16). The fertilizer was applied at the manufacturers' recommended concentration, followed by water to dissolve it and drainage into the soil. The system was the left undisturbed for 48 h prior to termination of experiment.

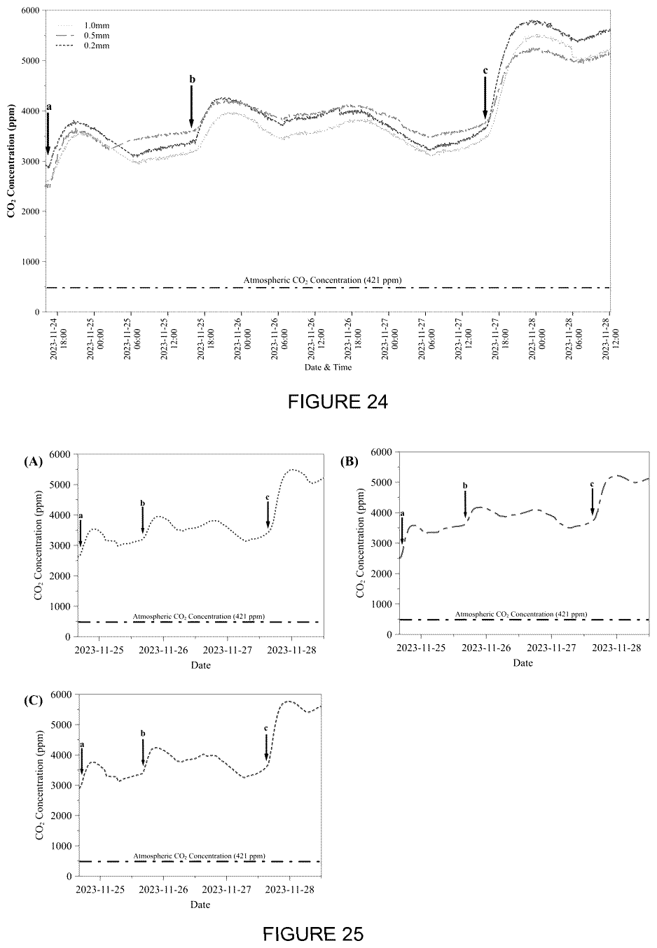

FIGS. 24 and 25 show the results. Dry soil had an average CO₂ concentration of ±2400 ppm with the addition of water (Arrow a) causing an increase to 3500-3700 ppm depending on membrane thickness. The second addition of water (Arrow b) resulted in an increase in CO₂ concentration to 4000-4200 ppm depending on membrane thickness. The addition of water (same volume as previously used) in conjunction with a water-soluble fertilizer (high in nitrogen and carbon) resulted in a rapid increase in CO₂ levels from ±3600 ppm to ±5800 ppm (Arrow c) where after the CO₂ concentrations remained at these elevated levels for more than 18 h post dosing. All the membranes performed equally as well as the control system, with slight variations.

FIG. 24 shows a graph of the gaseous CO₂ production in soil during water and fertilizer application, using various thicknesses of membrane-enclosed sensors ((Dotted line) 1 mm thick membrane, (Dash-dot line) 0.5 mm thick membrane, and (Dash line) 0.2 mm thick membrane). Atmospheric concentrations of CO₂ are indicated by the horizontal dash-dotted line (±421 ppm).

FIG. 25 shows a graph of the responsiveness to CO₂ levels of various thickness of membranes ((A) 1 mm thick membrane (B) 0.5 mm thick membrane and (C) 0.2 mm thick membrane) in untreated soil during water and fertilization with active pumping. Atmospheric concentrations of CO₂ are indicated by the horizontal dash-dotted line (±421 ppm).

### Trial 3

### Evaluation of Passive System Performance When Buried in Soil Containing Plants:

The aim of this experimental validation was to determine whether the passive version of the system could detect gaseous CO₂ produced in soil containing living plants.

The same experimental set-up relating to the sealing of the CO₂ sensors within a permeable membrane enclosure (as discussed above) was followed. The 0.5 mm-thick membranes were used in this experiment since previous trials showed good permeability and sufficient robustness and durability. A similar experimental set-up was followed for the burying of the apparatus or sensing units in the pots containing potting soil, except that each pot contained a single sensing unit. Three (3) pots were utilized, each representing a different treatment condition, namely (1) Plants, water and fertilizer, (2) Plants and water, and (3) No plants and water. The pots were placed under grow lights on a 12 h on and 12 h off cycle. A water-soluble fertilizer was used containing 112 g/kg N, 52 g/kg P, 264 g/kg K and 19 g/kg Mg (NPK+Mg=2:1:5+1). The fertilizer was applied as recommended by manufacturer, watered, and allowed to drench the soil of pot 1.

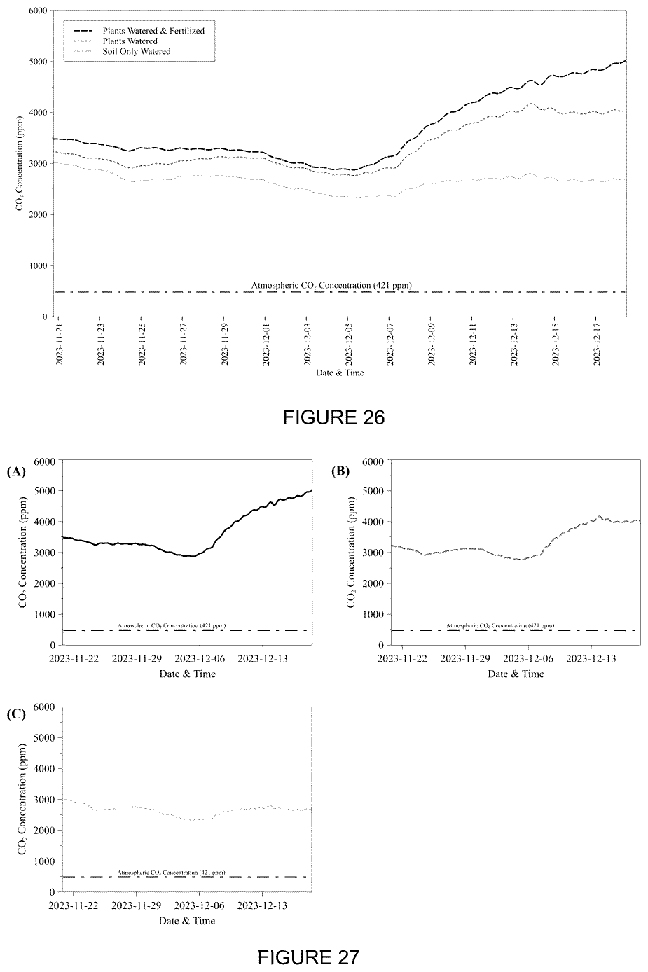

FIG. 26 shows the effect of living plants on CO₂ production in soil as monitored by the passive system, using 0.5 mm-thick membranes. All sensing units were buried 5-10 cm from the soil surface in individual pots containing the same homogenous soil. As indicated by the legend on the graph, the pot containing soil, plants, watered and fertilized is shown by the black dashed line, the pot containing soil, plants and watered is shown by the dark grey dotted line and the pot containing soil only and watered is shown by the light grey dash-dotted line. All trials were conducted simultaneously under the same experimental conditions with grow lights with a 12 h on and 12 h off cycle. Atmospheric concentrations of CO₂ are indicated by the horizontal dash-dotted line (±421 ppm).

FIG. 27 shows the effects of living plants on CO₂ production in soil as detected by the system using 0.5 mm-thick membranes. All sensing units were buried 5-10 cm from the soil surface in individual pots containing the same homogenous soil. The following is shown: (A) Pot containing soil, plants, watered and fertilized (Black solid line), (B) Pot containing soil, plants and watered (Dark grey dashed line) and (C) Pot containing soil only and watered (Light grey dotted line). All trials were conducted simultaneously under the same experimental conditions with grow lights with a 12 h on and 12 h off cycle. Atmospheric concentrations of CO₂ are indicated by the horizontal dash-dotted line (±421 ppm).

### Evaluation of Passive System Performance When Buried in Sterilized, Unsterilized and Sterilized Soil Amended With a Soil Microbe Mixture:

The aim of this experimental validation was to determine whether the passive version of the system could detect gaseous CO₂ produced in unsterilized, sterilized and sterile soil amended with a soil microbe mixture.

The same experimental set-up relating to the sealing of the CO₂ sensors within a permeable membrane enclosure was followed. The 0.5 mm-thick membranes were used in this experiment. A similar experimental set-up was followed for the burying of the sensing units or apparatuses in the pots containing potting soil, except that each pot contained a single sensing unit.

Three (3) pots were used, with each representing a different condition, namely (1) Sterilized soil amended with a commercial soil microbial mixture (2) Sterilized soil, and (3) Unsterilized soil. All soil used came from the same batch and the soil was sterilized 3 times (24 h apart) by autoclaving the soil in sealed containers. All pots were regularly watered to maintain optimal soil moisture.

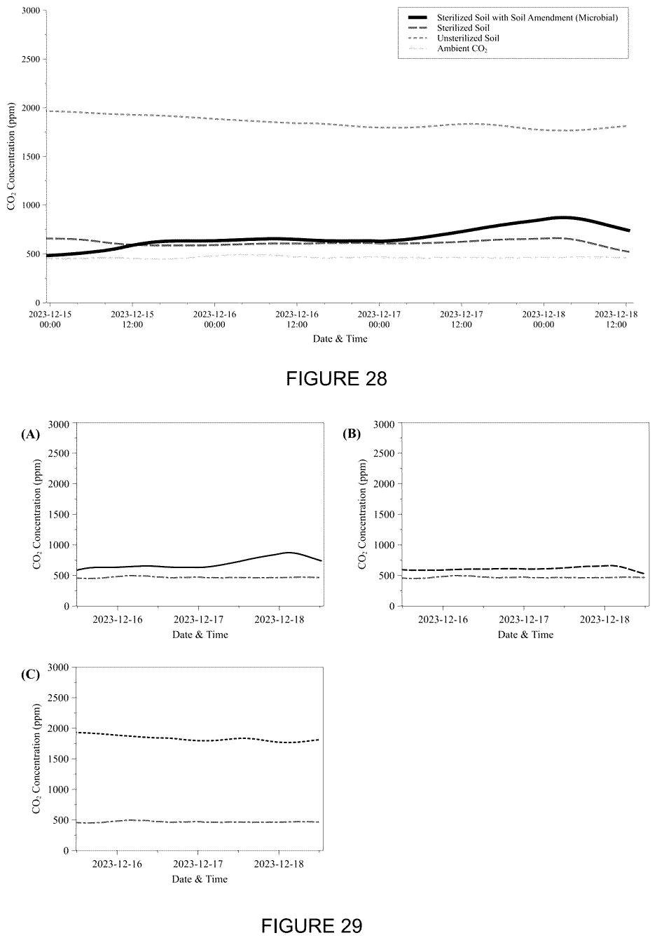

FIG. 28 demonstrates the effects of sterilized, sterilized with microbial amendment and unsterilized soil on CO₂ detected using sensing unit covered with 0.5 mm-thick membranes. All membranes were buried about 5-10 cm from the soil surface with 3 individual pots used, each containing its own sensing unit that has a membrane enclosing a CO₂ sensor, with all containing the same soil type (albeit sterilized or unsterilized). As show in the graph legend, Sterilized soil with microbial amendment (commercial microbial mixture) is shown in the black solid line, Sterilized soil is shown in the dark grey dashed line, Unsterilized soil is shown in the grey dotted line and ambient CO₂ levels is shown in the light grey dash-dot line. All pots were regularly watered to maintain optimal soil moisture.

FIG. 29 demonstrates the effects sterilized, sterilized with microbial amendment and unsterilized soil has on CO₂ production using 0.5 mm membranes. All membranes were buried about 5-10 cm from soil surface with 3 individual pots being used, each containing its own sensing unit with a membrane enclosing a CO₂ sensor, with all containing the same soil albeit sterilized or unsterilized. The following is shown: (A) Sterilized soil with microbial amendment (commercial microbial mixture) (black solid line), (B) Sterilized soil (black dash line), (C) Unsterilized soil (black dotted line) and ambient CO₂ levels (grey dash-dot line). All pots were regularly watered to maintain optimal soil moisture. Atmospheric concentrations of CO₂ are indicated by the horizontal dash-dotted line.

Advantageously, the apparatus and system described herein is capable of monitoring more than one soil parameter continuously. The apparatus includes an on-line carbon dioxide sensor for detecting and measuring carbon dioxide respiration. The accumulation of microbial cells and increased plant growth within a soil should result in an increased production and subsequent increased flux of carbon dioxide across the carbon-dioxide permeable membrane. Fluctuations of at least these parameters over time can be monitored continuously and analysed to establish a level of soil respiration in a valuable soil sample.

The foregoing description has been presented for the purpose of illustration; it is not intended to be exhaustive or to limit the invention to the precise forms disclosed. Persons skilled in the relevant art can appreciate that many modifications and variations are possible in light of the above disclosure.

The language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

Finally, throughout the specification and accompanying claims, unless the context requires otherwise, the word ‘comprise’ or variations such as ‘comprises’ or ‘comprising’ will be understood to imply the inclusion of a stated integer or group of integers but not the exclusion of any other integer or group of integers.

1. An apparatus for measuring concentration of a gas within soil comprising:
a tubular housing configured to extend beneath the soil, the tubular housing including:
an outer protective sleeve with perforations therein;
a support structure within the outer protective sleeve. the support structure defining an inner chamber;
a gas permeable membrane carried by the support structure and configured to prevent ingress of soil or liquid into the inner chamber; and
a gas sensor in communication with the inner chamber and configured to measure a concentration of the gas in the inner chamber.

2. The apparatus as claimed in claim 1, wherein the tubular housing includes an extension with a predetermined length, wherein the predetermined length is selected for monitoring gasses in a soil zone of interest including a compartment connected to the tubular housing, the compartment configured to project substantially above the soil in use.

3. The apparatus as claimed in claim 2, wherein the compartment has a width greater than a width of the tubular housing.

4. The apparatus as claimed in claim 2, including an extension piece which spaces the compartment and the inner chamber apart by a predetermined distance, and wherein the predetermined distance is selected for monitoring gasses in a soil zone of interest.

5. The apparatus as claimed in claim 4, wherein the extension piece has an outer wall that is not gas permeable.

6. The apparatus as claimed in claim 4, wherein the extension piece is in fluid communication with the inner chamber and with the compartment and is configured to allow gas diffusion from the inner chamber to the compartment.

7. The apparatus as claimed in claim 1, including a pump configured to move a carrier gas through the inner chamber to transport gas in the inner chamber to the gas sensor.

8. The apparatus as claimed in claim 7, wherein the support structure includes a conduit connected to the pump and wherein the conduit is configured to allow the flow of the carrier gas into the inner chamber.

9. The apparatus as claimed in claim 1, wherein the gas permeable membrane is a carbon dioxide permeable silicone membrane.

10. The apparatus as claimed in claim 1, wherein the gas sensor is configured to measure carbon dioxide (CO₂) levels.

11. The apparatus as claimed in claim 1, including a processing module configured to receive a signal from the sensor and process the signal to monitor the levels of carbon dioxide in the soil.

12. The apparatus as claimed in claim 1, wherein the gas sensor is a nondispersive infrared (NDIR) CO₂ sensor.

13. The apparatus as claimed in claim 1, wherein the gas sensor is an Internet of Things (IoT) sensor in wireless data communication with a network.

14. The apparatus as claimed in claim 1, wherein the tubular housing has a spiked end for insertion into the soil.

15. A system for monitoring soil respiration within soil comprising:
the apparatus as claimed in claim 1; and
a controller configured to be in data communication with the gas sensor of the apparatus to continuously monitor the soil respiration measured.

16. The apparatus as claimed in claim 2, wherein the compartment contains the gas sensor.

17. The apparatus as claimed in claim 2, wherein the compartment contains a processing module configured to receive a signal from the sensor and process the signal to monitor the levels of carbon dioxide in the soil.

18. The apparatus as claimed in claim 2, including an outlet in the compartment which provides an exit for gasses within the compartment to atmosphere.

19. The apparatus as claimed in claim 7, wherein the gas permeable membrane is silicone tubing enclosed within the housing.

20. The apparatus as claimed in claim 19, wherein the tubing is coiled within the housing and is held in place by a tubing support frame.
